(19) United States
(12) Patent Application Publication
(10) Pub. No.: US 2023/0181300 A1
(43) Pub. Date: Jun. 15, 2023

(54) SMART TOOTH CLEANING DEVICE

(71) Applicant: National Taiwan University of Science and Technology, Taipei (TW)

(72) Inventors: Jeng-Ywan Jeng, Taipei (TW); Zheng-Ye She, Taipei (TW); Cheng-Yi Tsai, Taipei (TW); Azam Hamza, Taipei (TW); Ajeet Kumar, Taipei (TW)

(21) Appl. No.: 17/742,391

(22) Filed: May 11, 2022

(30) Foreign Application Priority Data
Dec. 9, 2021 (TW) 110145990

(51) Int. Cl. A61C 17/22; A46B 15/00

(52) U.S. Cl. CPC A61C 17/221; A46B 15/0036; A46B 15/0038; A46B 2200/1066

(57) ABSTRACT

The present invention provides a smart tooth cleaning device comprises a hand piece having a bearing surface, a tooth cleaning piece, a driving module connected to the tooth cleaning piece for driving the tooth cleaning piece to move, a first camera module comprising a first optical lens and a first image capturing element, and a first light source module comprising a plurality of light-emitting elements. The tooth cleaning piece has a tooth cleaning body, a brush head, and a plurality of bristles. The first camera module is disposed on the bearing surface and is electrically connected to the control module. The first image capturing element receives light along an optical axis of the first optical lens. A first angle θ1 between the optical axis and a first direction, is in a range of 5°to 30°. The first light source module is disposed on the bearing surface.

### CROSS REFERENCE TO RELATED APPLICATIONS

The present application claims the benefit of Taiwanese Patent Application No. 110145990 filed on Dec. 9, 2021, the contents of which are incorporated herein by reference in their entirety.

### TECHNICAL FIELD

The present invention relates to the technical field of dental health care appliances, in particular to a smart tooth cleaning device that illuminates the oral cavity from the outside and captures images of the oral cavity and teeth.

### BACKGROUND

Tooth cleaning greatly impacts on personal health, and the most basic habit of tooth cleaning is to brush the teeth every morning and evening. In recent years, the number of people suffering from oral cancer has gradually increased. One of reasons is a wrong way of brushing the teeth. The wrong way of brushing the teeth easily causes damage to the oral cavity and teeth, thereby making bacteria or pathogens in the oral cavity enter the human body through wounds. In addition to increasing the risk of oral cancer, it will also increase the risk of cancer in other organs. Therefore, in addition to developing correct tooth brushing habits, the health of the oral cavity and teeth needs to be regularly checked. In addition to timely detection of oral lesions, the health of the oral cavity and teeth may also be recorded and monitored at any time.

In order to observe and record the states of the oral cavity and teeth at any time, the industry proposes a technical solution of installing a camera module and a light source on a brush head of a toothbrush, as disclosed in the Taiwan invention patent 1738529. However, in the Taiwan invention patent 1738529, a camera lens and the light source are disposed on the brush head. Although the camera lens may go deep into the oral cavity to capture images of the oral cavity and teeth, since the camera lens and the light source are located at the bottom of bristles, they are easily blocked by the bristles and foam of toothpaste and images with a smaller viewing angle may only be captured, which is not conducive to a complete observation of the states of the overall oral cavity and teeth.

### SUMMARY

In view of this, an objective of the present invention is to provide a smart tooth cleaning device. A camera module and a light-emitting module are disposed at the end of the hand piece facing the tooth cleaning piece, so that images of the oral cavity and teeth are captured from the outside of the oral cavity, and a camera device is not blocked by bristles or foam of toothpaste and may capture complete images of the oral cavity and teeth.

An embodiment of a smart tooth cleaning device includes a hand piece, a tooth cleaning piece, a driving module, a first camera module, and a first light source module. The hand piece includes a first end and a second end, and the first end has a bearing surface. The tooth cleaning piece has a tooth cleaning body, a brush head and a plurality of bristles, the tooth cleaning body is detachably bonded to the bearing surface, the bristles are disposed on the brush head, the tooth cleaning body extends along a first direction, and the brush head is disposed at the end of the tooth cleaning body. The driving module is disposed in the hand piece and connected to the tooth cleaning piece, and the drive module drives the tooth cleaning piece to move. A control module is electrically connected to the driving module. The first camera module is disposed on the bearing surface and electrically connected to the control module, the first camera module includes at least a first optical lens and at least a first image capturing element, the first image capturing element receives light along the optical axis of the first optical lens, there is a first angle 61 between the optical axis and the first direction, and the first angle 61 is in a range of 5° to 30°. The first light source module is disposed on the bearing surface and includes a plurality of light-emitting elements.

In another embodiment, there is a second angle 62 between a normal line of the bearing surface and the first direction.

In another embodiment, the second angle 62 is equal to the first angle 61, and the optical axis is parallel to the normal line of the bearing surface.

In another embodiment, the second angle 62 is not equal to the first angle 61.

In another embodiment, the hand piece further comprises an accommodation recess, the accommodation recess is disposed on the bearing surface, and the first camera module is disposed on the accommodation recess.

In another embodiment, the smart tooth cleaning device provided by the present invention further includes an adjustment mechanism arranged on the hand piece and connected to the first camera module, and the adjustment mechanism makes the first camera module rotate along a second direction to adjust the first angle 01.

In another embodiment, the light-emitting elements surround the first camera module and are spaced apart from each other with equal angular distances.

In another embodiment, the light-emitting elements include first light-emitting elements that emit light with a first wavelength and a second light-emitting element that emits light with a second wavelength.

In another embodiment, the first wavelength, λ1 is in a range of 360 nm to 830 nm, and the second wavelength λ2 is in a range of ≧330 nm or ≧15 µm.

In another embodiment, the smart tooth cleaning device provided by the present invention further includes a blood detection module disposed on the hand piece, and the blood detection module detects the blood of a user holding the hand piece and generates detection data.

In another embodiment, the driving module includes a driver and a transmission element, the transmission element extends outward from the first end of the hand piece and is connected with the driver and the tooth cleaning piece, and a torque force of the driver is transmitted to the tooth cleaning piece through the transmission element, so as to make the tooth cleaning piece rotate or vibrate.

In another embodiment, the smart tooth cleaning device provided by the present invention further includes a second camera module and a second light source module that are arranged on the transmission element and correspond to the brush head, the second camera module includes a plurality of second optical lenses and a plurality of second image capturing elements, the second light source module includes a plurality of second light-emitting elements, the brush head is provided with a plurality of transparent windows, and the second optical lenses and the second light-emitting elements correspond to the transparent windows in position.

In another embodiment, the smart tooth cleaning device provided by the present invention further includes the control module, the control module includes a processor and a wireless transceiver, the processor is electrically connected to the driving module, the first camera module and the first light source module and controls the driving module, the first camera module and the first light source module to be actuated, and the wireless transceiver receives a signal from an external electronic device or transmits data to the external electronic device.

In the smart tooth cleaning device provided by the present invention, the first camera module and the first light source module are disposed on the bearing surface of the first end of the hand piece, the first angle of 5° to 30° is formed between the optical axis of the first camera module and the first direction along which the tooth cleaning body extends, and the first optical lens adopts a wide-angle lens, so the images of the oral cavity and teeth may be captured from the outside of the oral cavity, blocking by the bristles and the foam of toothpaste is avoided, and the images of the complete oral cavity and teeth may be captured. In addition to emitting visible light for illumination, the first light source module may also emit near-ultraviolet light or far-infrared light, so as to detect lesions of the oral cavity or treat affected parts of the oral cavity.

### BRIEF DESCRIPTION OF THE DRAWINGS

FIG. 1 is a three-dimensional view of an embodiment of a smart tooth cleaning device provided by the present invention.

FIG. 2 is a three-dimensional exploded view of the smart tooth cleaning device in FIG. 1.

FIG. 3 is a side view of the smart tooth cleaning device in FIG. 1.

FIG. 4 is a front view of the smart tooth cleaning device in FIG. 1.

FIG. 5 is an exploded view of FIG. 4.

FIG. 6 is a system block diagram of the smart tooth cleaning device in FIG. 1.

FIG. 7 is a partial three-dimensional view of another embodiment of the smart tooth cleaning device provided by the present invention.

### DETAILED DESCRIPTION OF THE EMBODIMENTS

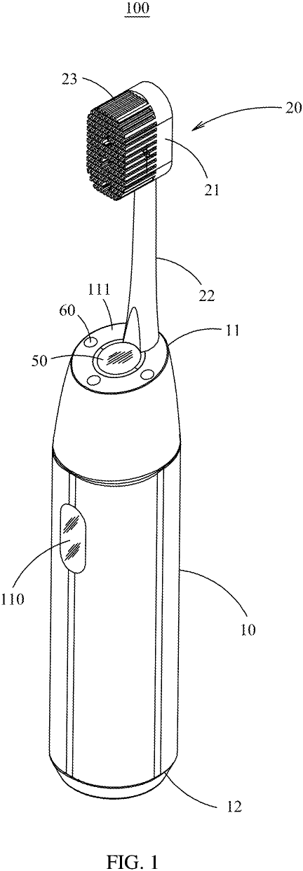

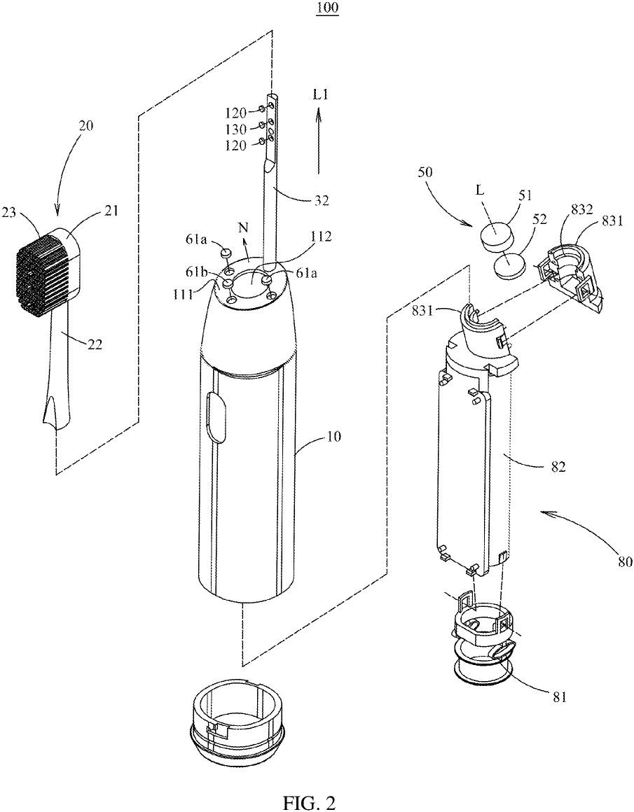

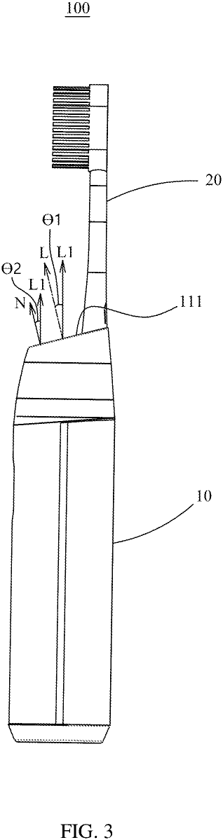

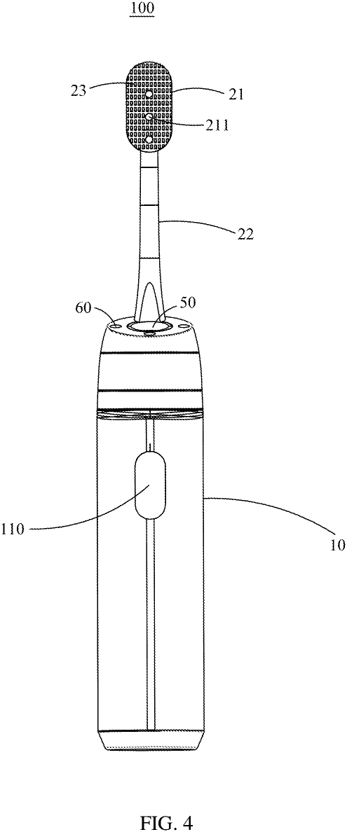

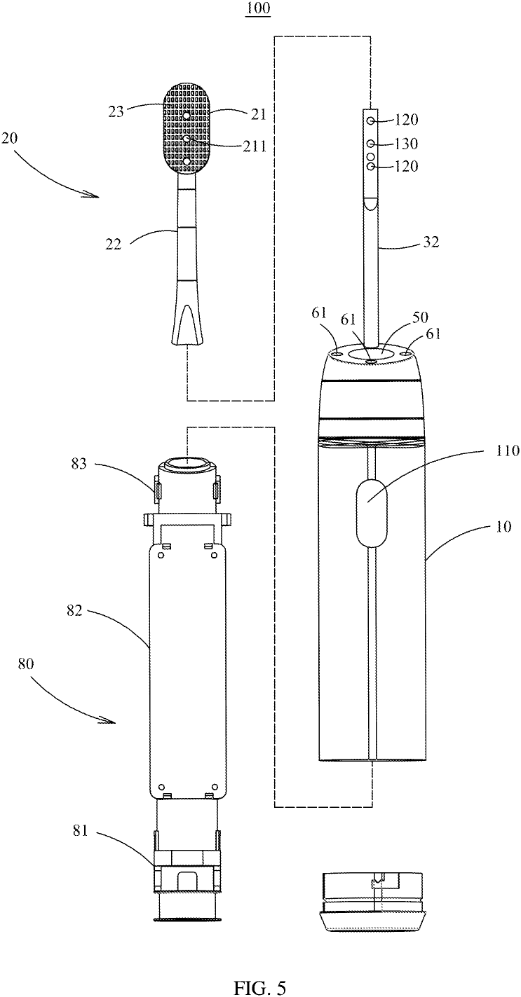

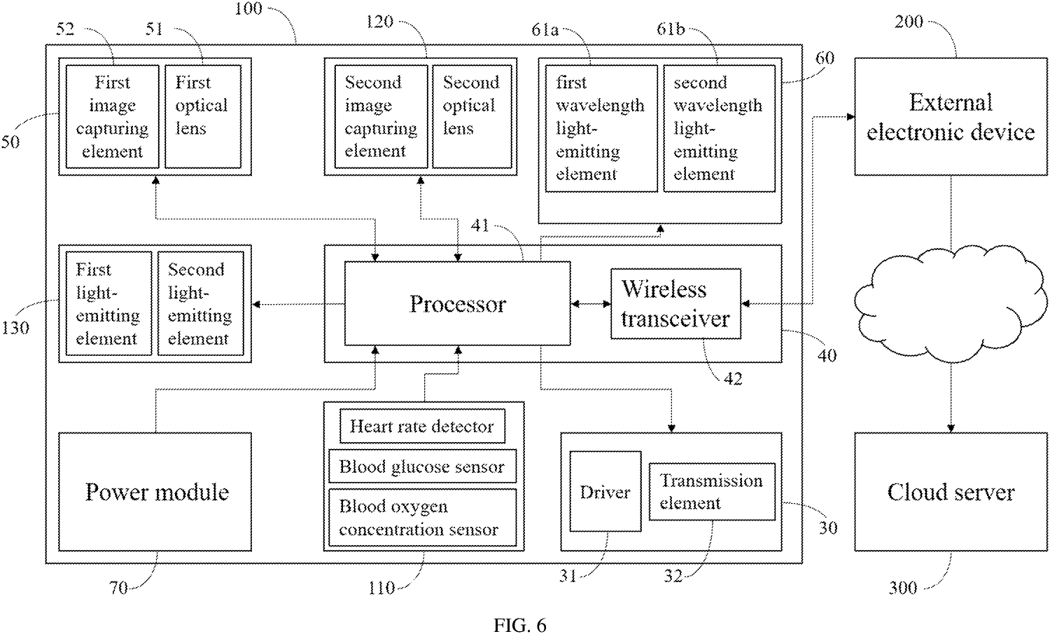

Referring to FIG. 1, FIG. 2, FIG. 3, FIG. 4, FIG. 5, and FIG. 6, which represents an embodiment of the smart tooth cleaning device provided by the present invention. A smart tooth cleaning device 100 provided by this embodiment includes a hand piece 10, a tooth cleaning piece 20, a driving module 30, a control module 40, a first camera module 50, and a first light source module 60.

As shown in FIG. 1, the hand piece 10 has a cylindrical shell, which, for example, may be circular and cylindrical or elliptical and cylindrical, and allows a hand of a user to hold. The hand piece 10 has a first end 11 and a second end 12, and the first end 11 and the second end 12 are located at two axial ends of the cylindrical shell. The second end 12 of the hand piece 10 may be placed on a bearing seat for storage and charging. The first end 11 of the hand piece 10 has a bearing surface 111. The center of the bearing surface 111 has a recess 112 configured to accommodate the first camera module 50, and a detailed structure will be described in the following paragraphs.

The tooth cleaning piece 20 includes a brush head 21 , a tooth cleaning body 22 and a plurality of bristles 23, and the tooth cleaning body 22 is connected to the brush head 21 and is detachably sleeved on an aftermentioned transmission element 32 disposed on the bearing surface 111. That is, the tooth cleaning body 22 is internally provided with a bonding hole, the aftermentioned transmission element 32 of the driving module 30 is arranged in the bonding hole in a penetration manner, and the bottom of the tooth cleaning body 22 is in contact with the bearing surface 111, so that the tooth cleaning body 22 is supported by the hand piece 10 and located on the bearing surface 111 of the first end 11. The bristles 23 are arranged on the brush head 21. The tooth cleaning body 22 may match in shape with a holding part, and the tooth cleaning body 22 may be made of an elastic and flexible material, so that the tooth cleaning piece 20 may easily generate an aftermentioned rotation or vibration to clean the teeth. The arrangement shape of the bristles 23 may be configured by matching with the arrangement shape of the brush head 21.

The driving module 30 includes a driver 31 and the transmission element 32, the driver 31 is disposed in the shell of the hand piece 10, the transmission element 32 is disposed in the shell and connected to the driver 31, and the transmission element 32 passes through the bearing surface 111 of the first end 11 of the hand piece 10 to extend to the outside of the shell. The transmission element 32 in this embodiment passes through the bearing surface 111 from a position close to an edge of the bearing surface 111. The transmission element 32 in this embodiment is a rod body, which extends from the bearing surface 111 to the first direction L1 away from the hand piece 10. In this embodiment, the first direction L1 is the same as an axial direction of the cylindrical shell of the hand piece 10, but this embodiment is not limited to this, and the first direction L1 may also form an angle of greater than 0° with the axial direction of the cylindrical shell, for example, the transmission element 32 is slightly inclined towards the center of the bearing surface 111 or inclined towards a direction away from the center of the bearing surface 111, depending on design requirements. Since the tooth cleaning body 22 of the tooth cleaning piece 20 is sleeved on the transmission element 32, the tooth cleaning body 22 also extends along the first direction L1. In this embodiment, the driver 31 is an electric motor, the transmission element 32 is the rod body, and the driver 31 outputs a torsion force and converts it into rotation of the transmission element 32 or vibration orthogonal to an extension direction of the transmission element 32, thereby making the tooth cleaning piece 20 rotate or vibrate to clean the teeth.

The control module 40 is disposed inside the cylindrical shell of the hand piece 10, the control module 40 includes a circuit board (not shown), a processor 41 and a wireless transceiver 42, and the processor 41 and the wireless transceiver 42 are disposed on the circuit board and form an electrical connection through the circuit board. The processor 41 is electrically connected to the driving module, the first camera module and the first light source module, and the wireless transceiver 42 receives a signal from an external electronic device or transmits data to the external electronic device (as shown in FIG. 5). The processor 41 may be a commercially available microprocessor, and the wireless transceiver 42 may be a wireless communication chip set conforming to various communication protocols.

The control module 40 may also be connected to a power module 70, and the power module 70 includes a transformer circuit, a rectifier circuit, and a lithium battery or other electrical energy storage assemblies, and is configured to provide power to the driving module 30, the control module 40, the first camera module 50 and the first light source module 60.

The smart tooth cleaning device 100 provided by this embodiment further includes a bearing module 80, and the bearing module 80 is disposed inside the cylindrical shell of the hand piece 10, and is configured to bear the driving module 30, the control module 40 and the first camera module 50. The bearing module 80 includes a first bearing part 81, a second bearing part 82 and a third bearing part 83, the first bearing part 81 is disposed on the second end 12 of the hand piece 10 and is located at the bottom of the hand piece 10, and the driver 31 of the driving module 30 is born on the first bearing part 81. The second bearing part 82 is connected to the first bearing part 81. In this embodiment, the second bearing part 82 is connected with the first bearing part 81 in a clamped manner, and the second bearing part 82 bears the control module 40 and the power module 70. The third bearing part 83 is connected to the second bearing part 82 and is close to the first end 11 of the cylindrical shell of the hand piece 10, the third bearing part 83 includes two semi-cylindrical shells 831, and is configured to bear the first camera module 50, the first camera module 50 is accommodated in a cylindrical accommodation space formed by the third bearing part 83, and an opening 832 is formed in one end of the third bearing part 83, so that external light passes through the recess 112 of the bearing surface 111 and the opening 832 and then is received by the first camera module 50 to generate images.

The first camera module 50 includes a first optical lens 51 and a first image capturing element 52, the first optical lens 51 is held by the third bearing part 83, and the first image capturing element 52 is disposed below the first optical lens 51 and receives light along an optical axis L of the first optical lens 51. There is a first angle 61 between the optical axis L of the first optical lens 51 in this embodiment and the first direction L1 along which the tooth cleaning body 22 of the tooth cleaning piece 20 extends, and the first angle 61 is in a range of 5° to 30°. The first angle 61 between the optical axis L and the first direction L1 is achieved by inclining the bearing surface 111 with respect to the first direction L1. In addition to the hand piece 10 having an angle the same as the first angle 61 in this embodiment, the first optical lens 51 in this embodiment is a wide-angle lens with an ultra-wide angle, and its viewing angle may reach nearly 180°. There is a second angle 62 between a normal line N of the bearing surface 111 in this embodiment and the first direction L1. Since the optical axis L of the first optical lens 51 in this embodiment is parallel to the normal line N of the bearing surface 111, the first angle 61 and the second angle 62 are same.

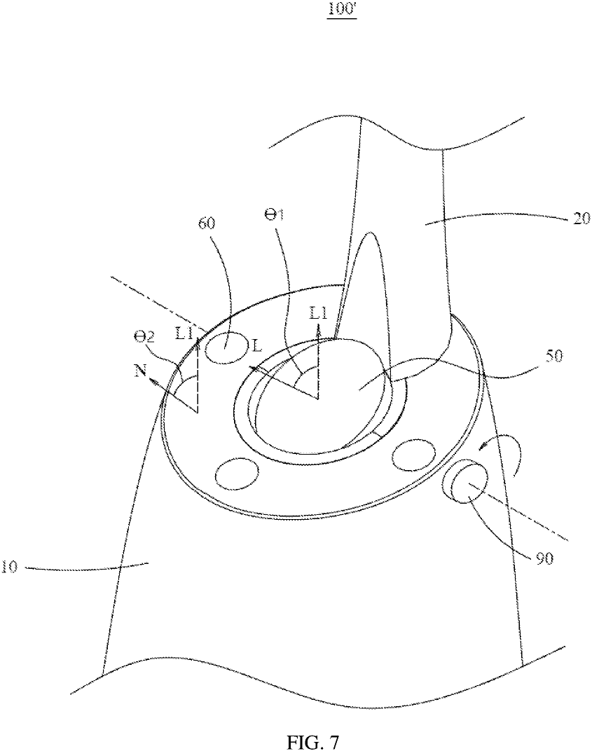

FIG. 7 shows another embodiment of the smart tooth cleaning device provided by the present invention. This embodiment has a part of the same structure as the previous embodiment, and the same assemblies are given the same symbols and their descriptions are omitted. A smart tooth cleaning device 100′ provided by this embodiment further includes an adjustment mechanism 90, the first camera module 50 is disposed on the bearing module 80 through the adjustment mechanism 90, and the adjustment mechanism 90 in this embodiment includes a rotating shaft, a friction washer and a knob, the rotating shaft is arranged on the first camera module 50 in a penetration manner, the friction washer is disposed between the rotating shaft and the bearing module 80, and the knob is disposed outside the shell of the hand piece 10 and connected to the rotating shaft. The user may rotate the rotating shaft by clamping the knob with fingers to make the first camera module 50 rotate, and the friction washer provides a high frictional force to make the rotating shaft located at any angle. In this embodiment, by disposing the adjustment mechanism 90, the angle of the optical axis L of the first optical lens 51 of the first camera module 50 may be changed arbitrarily, so that the first angle 61 between the optical axis L and the first direction L1 is variable; and the second angle 62 between the normal line N of the bearing surface 111 and the first direction L1 is constant, so during the rotation of the first camera module 50, the first angle 61 may be the same as or different from the second angle 62. Since the first optical lens 51 in this embodiment may be rotated, the first optical lens 51 may be the wide-angle lens or a general conventional lens. In addition, the normal line N of the bearing surface 111 in this embodiment may also be parallel to the first direction L1, and it is only necessary to rotate the first camera module 50 to adjust the first angle 61 between the optical axis L and the first direction L1. The adjustment mechanism 90 is not limited to a structure of this embodiment, and may also be rotated electronically.

Referring to FIG. 1 to FIG. 6 again, the first light source module 60 is disposed on the bearing surface 111 of the first end 11 of the hand piece 10. The first light source module 60 includes a plurality of light-emitting elements 61. The plurality of first light-emitting elements 61 are disposed around the recess 112 of the bearing surface 111, and the plurality of first light-emitting elements 61 in this embodiment are spaced apart from each other with equal angular distances. The plurality of first light-emitting elements 61 include first wavelength light-emitting elements 61a capable of emitting light with a first wavelength λ1 and a second wavelength light-emitting element 61b capable of emitting light with a second wavelength λ2. The first wavelength λ1 is in a range of 360 nm to 830 nm, and the second wavelength λ2 is in a range of ≦330 nm or ≧15 µm. The first wavelength λ1 is visible light, so the first wavelength light-emitting elements 61a are configured to illuminate the oral cavity, and the first image capturing element 52 generates an image of the visible light after receiving the visible light. The second wavelength λ2 may be far-infrared light or near-ultraviolet light, which may be configured to illuminate the oral cavity to obtain special images of lesions of the oral cavity. For example, an image of distribution of dental plaque on the teeth is obtained by the near-ultraviolet light, or a thermal image of temperature distribution of the oral cavity is obtained by the infrared light. Therefore, the first camera module 50 in this embodiment may also include a plurality of first optical lenses 51 and a plurality of first image capturing elements 52, which are respectively configured to capture light with different wavelengths to generate visible light images or other special images. A light-emitting direction of the first light-emitting elements 61 may be along the normal line N of the bearing surface 111 or along a direction intersected with the optical axis L of the first optical lens 51.

This range of wavelengths are used to recognize the images, in addition to detecting the topographies of the lesions in a visible light range, such as: gingivitis, periodontal disease, tongue ulcers, coated tongue, intraoral epidermal tissue ulcers and bacterial infections, lip ulcers and bacterial infections, oral mucositis, tooth decay, sensitive teeth, dental calculus, tea residues, smoking spots, betel nut residues, wisdom teeth, plaque, oral cleanliness, toothbrushing certainty, oral cancer, malocclusion and gum bleeding. The infrared light is capable of easily penetrating tooth enamel, so the lesions that are not easily detected by the visible light may be detected. For example, initial tooth decay and sensitive teeth may be further detected by using near-infrared light, so as to increase the possibility of detecting various lesions and improve the accuracy of lesion detection. In addition, the far-infrared light may also be used as a phototherapy for treating the affected parts of the oral cavity. The control module 40 may control the first wavelength light-emitting elements 61a and the second wavelength light-emitting element 61b to emit the light in different modes of use.

In addition, the surface of the shell of the hand piece 10 will be in contact with the skin and hands of the user because the user holds it with the palm. The smart tooth cleaning device 100 provided by this embodiment further includes a physiological detection module 110, and the physiological detection module 110 includes a plurality of physiological sensors, such as a heart rate sensor, a blood glucose sensor, a blood oxygen concentration sensor, etc. When the user holds the hand piece 10, the plurality of physiological sensors may detect the pulse by or without contacting with the skin to measure the heart rate, or measure the blood glucose level or the blood oxygen concentration by enabling the light to pass through the veins.

As shown in FIG. 5, the smart tooth cleaning device 100 provided by the present invention may be connected with the external electronic device 200 through wireless communication, the images captured by the first camera module 50 may be transmitted to the external electronic device 200, such as a smartphone, a tablet computer or a laptop, for storage through the wireless transceiver 42 of the control module 40, while the external electronic device 200 may also transmit various set values to the smart tooth cleaning device 100 through an installed application. Additionally, the external electronic device 200 may also upload image data of the oral cavity or teeth or measured heart rate data, blood glucose or blood oxygen concentration data received from the smart tooth cleaning device 100 to a cloud server 300 for the convenience of analysis of big data.

The smart tooth cleaning device 100 provided by the present invention further includes a second camera module 120 and a second light source module 130, and the second camera module 120 and the second light source module 130 are disposed on the transmission element 32 of the driving module 30 and correspond to the brush head 21 of the tooth cleaning piece 20. The second camera module 120 includes a plurality of second optical lenses and a plurality of second image capturing elements, the second light source module 130 includes a plurality of second light-emitting elements, the brush head 21 is provided with a plurality of transparent windows 211, the second optical lenses and the second light-emitting elements correspond to the transparent windows 211 in position, light emitted by the second light-emitting elements is emitted to the outside through the transparent windows 211, and light reflected from the oral cavity and teeth also enters the second camera module 120 through the transparent windows 211 to generate images. The plurality of second light-emitting elements of the second light source module 130 may emit light in the visible light wavelength range and light outside the visible light range, such as the far-infrared light, the near-ultraviolet light, etc., while the plurality of second image capturing elements of the second camera module 120 may also receive light with different wavelengths to generate different images.

In another embodiment, a storage space for a dentifrice may be disposed on the tooth cleaning piece 20. In cooperation with the oral images captured by the aforementioned first camera module 50 and second camera module 120, when the tooth cleaning piece 20 moves to a position of tooth cleaning needed to be enhanced particularly, for example, the teeth having the dental plaque or the particularly yellow teeth, the processor 41 controls a micropump disposed on the hand piece 10 to increase pressure to squeeze out the dentifrice from the tooth cleaning piece 20 to eliminate the dental plaque or enhance cleaning.

In the smart tooth cleaning device provided by the present invention, the camera module and the light source module are disposed on the bearing surface of the first end of the hand piece, the first angle of 5° to 30° is formed between the optical axis of the camera module and the first direction along which the tooth cleaning body extends, and the optical lens adopts the wide-angle lens, so the images of the oral cavity and teeth may be captured from the outside of the oral cavity, blocking by the bristles and the foam of toothpaste is avoided, and the images of the complete oral cavity and teeth may be captured. In addition to emitting the visible light for illumination, the light source module may also emit the near-ultraviolet light or the far-infrared light, so as to detect the lesions of the oral cavity or treat the affected parts of the oral cavity.

However, the above are merely the preferred embodiments of the present invention, and should not limit the scope of the present invention, that is, simple equivalent changes and modifications made according to the scope of the present invention and the description of the present invention all still fall within the scope covered by the patent of the present invention. In addition, any embodiment of the present invention or the scope of the patent application need not achieve all objectives, advantages or features disclosed by the present invention. Moreover, the abstract and the title are merely intended to assist in the search for patent documents and are not intended to limit the scope of the rights of the present invention. Furthermore, the terms such as “first” and “second” mentioned in this description or the scope of the patent application are merely used to name the elements or to distinguish different embodiments or ranges, and are not used to limit the upper or lower limit of the number of elements.

### DESCRIPTION OF SYMBOLS

| | |
|---|---|
| 10 | hand piece |
| 11 | first end |
| 12 | second end |
| 20 | tooth cleaning piece |
| 21 | brush head |
| 22 | tooth cleaning body |
| 23 | bristles |
| 30 | driving module |
| 31 | driver |
| 32 | transmission element |
| 40 | control module |
| 41 | processor |
| 42 | wireless transceiver |
| 50 | first camera module |
| 51 | optical lens |
| 52 | first image capturing element |
| 60 | first light source module |
| 61 | first light-emitting element |
| 61a | first wavelength light-emitting element |
| 61b | second wavelength light-emitting element |
| 70 | power module |
| 80 | bearing module |
| 81 | first bearing part |
| 82 | second bearing part |
| 83 | third bearing part |
| 90 | adjustment mechanism |
| 100 | smart tooth cleaning device |
| 100′ | smart tooth cleaning device |
| 110 | physiological detection module |
| 111 | bearing surface |
| 112 | recess |
| 120 | second camera module |
| 130 | second light source module |
| 200 | external electronic device |
| 211 | transparent window |
| 300 | cloud server |
| 831 | shell |
| 832 | opening |
| L | optical axis |
| L1 | first direction |
| N | normal line |
| θ1 | first angle |
| θ2 | second angle |

1. A smart tooth cleaning device, comprising:
a hand piece, comprising a first end and a second end, wherein the first end has a bearing surface;
a tooth cleaning piece, having a tooth cleaning body, a brush head, and a plurality of bristles, wherein the tooth cleaning body is detachably bonded to the bearing surface, the bristles are disposed on the brush head, the tooth cleaning body extends along a first direction, and the brush head is disposed at the end of the tooth cleaning body;
a driving module, disposed in the hand piece and connected to the tooth cleaning piece, wherein the drive module drives the tooth cleaning piece to move;
a first camera module, disposed on the bearing surface and electrically connected to a control module, wherein the first camera module comprises at least a first optical lens and at least a first image capturing element, the first image capturing element receives light along the optical axis of the first optical lens, there is a first angle θ1 between the optical axis and the first direction, and the first angle θ1 is in a range of 5° to 30°; and
a first light source module, disposed on the bearing surface and electrically connected to the control module, wherein the first light source module comprises a plurality of light-emitting elements.

2. The smart tooth cleaning device according to claim 1, wherein there is a second angle θ2 between a normal line of the bearing surface and the first direction, and the second angle θ2 is greater than 0°.

3. The smart tooth cleaning device according to claim 2, wherein the second angle θ2 is equal to the first angle θ1, and the optical axis is parallel to the normal line of the bearing surface.

4. The smart tooth cleaning device according to claim 1, wherein the hand piece further comprises an accommodation recess, the accommodation recess is disposed on the bearing surface, and the first camera module is disposed on the accommodation recess.

5. The smart tooth cleaning device according to claim 2, further comprising an adjustment mechanism, arranged on the hand piece and connected to the first camera module, wherein the adjustment mechanism makes the first camera module rotate along a second direction to adjust the first angle θ1.

6. The smart tooth cleaning device according to claim 3, further comprising an adjustment mechanism, arranged on the hand piece and connected to the first camera module, wherein the adjustment mechanism makes the first camera module rotate along a second direction to adjust the first angle θ1.

7. The smart tooth cleaning device according to claim 4, further comprising an adjustment mechanism, arranged on the hand piece and connected to the first camera module, wherein the adjustment mechanism makes the first camera module rotate along a second direction to adjust the first angle θ1.

8. The smart tooth cleaning device according to claim 1, wherein the light-emitting elements surround the first camera module and are spaced apart from each other with equal angular distances.

9. The smart tooth cleaning device according to claim 1, wherein the light-emitting elements comprise first light-emitting elements that emit light with a first wavelength and a second light-emitting element that emits light with a second wavelength.

10. The smart tooth cleaning device according to claim 1, wherein a first wavelength λ1 is in a range of 360 nm to 830 nm, and a second wavelength λ2 is in a range of ≦ 330 nm or ≧ 15 µm.

11. The smart tooth cleaning device according to claim 1, further comprising a physiological detection module, disposed on the hand piece, wherein the physiological detection module detects the body of a user holding the hand piece and generates detection data.

12. The smart tooth cleaning device according to claim 1, wherein the driving module comprises a driver and a transmission element, the transmission element extends outward from the first end of the hand piece and is connected with the driver and the tooth cleaning piece, and a torque force of the driver is transmitted to the tooth cleaning piece through the transmission element, so as to make the tooth cleaning piece rotate or vibrate.

13. The smart tooth cleaning device according to claim 12, further comprising a second camera module and a second light source module, wherein the second camera module and the second light source module are arranged on the transmission element and correspond to the brush head, the second camera module comprises a plurality of second optical lenses and a plurality of second image capturing elements, the second light source module comprises a plurality of second light-emitting elements, the brush head is provided with a plurality of transparent windows, and the second optical lenses and the second light-emitting elements correspond to the transparent windows in position.

14. The smart tooth cleaning device according to claim 1, further comprising the control module, wherein the control module comprises a processor and a wireless transceiver, the processor is electrically connected to the driving module, the first camera module and the first light source module and controls the driving module, the first camera module and the first light source module to be actuated, and the wireless transceiver receives a signal from an external electronic device or transmits data to the external electronic device.
